(19) United States
(12) Patent Application Publication
(10) Pub. No.: US 2021/0076882 A1
(43) Pub. Date: Mar. 18, 2021

(54) Experiential Cosmetic Product Dispensing Device

(71) Applicant: Sebastien Fauconnier, Neuvy Saint Se-Pulchre (FR)

(72) Inventor: Sebastien Fauconnier, Neuvy Saint Se-Pulchre (FR)

(21) Appl. No.: 16/959,566

(22) PCT Filed: Dec. 27, 2018
(86) PCT No.: PCT/FR2018/053553

§ 371 Date: Jul. 1, 2020

(30) Foreign Application Priority Data
Jan. 2, 2018 (FR) 1850014

(51) Int. Cl. A47K 5/12; A61L 9/14

(52) U.S. Cl. CPC A47K 5/1217; A47K 5/1204; A61L 9/14; A61L 2209/134; A61L 2209/132; A61L 2209/133; A61L 2209/111

(57) ABSTRACT

A cosmetic product dispensing device, which includes: a dispensing mechanism; at least one presence sensor; and a hand warmer associated with a heater. The presence sensor is configured to detect at least: a first type of hand presence, and a second type of hand presence, it further includes electronic management configured to send: a dispensing instruction to the dispensing mechanism, when the first presence type is detected, or a preheating instruction to the heater means, when the second presence type is detected.

### CROSS-REFERENCE TO RELATED APPLICATIONS

This Application is a Section 371 National Stage Application of International Application No. PCT/FR2018/053553, filed Dec. 27, 2018, the content of which is incorporated herein by reference in its entirety, and published as WO 2019/135045 on Jul. 11, 2019, not in English.

### FIELD OF THE DISCLOSURE

The field of the invention is that of the design and manufacture of cosmetic product dispensing devices. More specifically, the invention relates to a mode of activating the dispensing of a cosmetic product.

### BACKGROUND OF THE DISCLOSURE

In the field of the invention, liquid or semi-liquid cosmetic product dispensers are known. For example, soap dispensers are known which are periodically refilled with a stock of product ready to be dispensed.

The dispensers can be automated to enable dispensing of a dose of cosmetic product when a person positions their hand under a dispensing nozzle.

Such dispensers thus comprise:
a dispensing mechanism including a dispensing nozzle through which the cosmetic product is dispensed, and
a presence sensor for detecting a hand and controlling the dispensing mechanism to dispense the cosmetic product.

Such dispensers simplify the dispensing of product doses thanks to the contactless activation thereof.

Cosmetic preparation formulation and dispensing apparatuses are also known, intended to be installed directly in private individuals' homes, particularly for specific applications such as baby massage for example. Such apparatuses, particularly described in the patent documents EP 1 671 568, US 2011/303695, and WO 2006/134314 also have contactless activation.

However, the baby massage application requires specific execution conditions such as the need for the user, in this case the masseur or masseuse, to have their hands at a temperature close to that of the baby's body, especially to prevent a cold sensation for the latter.

For this, it is necessary for the user to be able to warm their hands, without for all that being away from the baby, which makes it possible to keep constant interaction with the latter and increases the baby's safety, particularly by limiting the risk of the baby falling off the table.

Yet, at the present time, there is no satisfactory solution meeting this requirement.

### SUMMARY

There is thus a need aimed at improving the circumstances for applying a dose of cosmetic product obtained via a dispenser.

This need is particularly clear for babies on whom a cosmetic product is to be applied in the form of massage. Indeed, the application of a product may be upsetting and even unpleasant for a child, particularly if the masseur's or masseuse's hands are at a lower temperature than that of the baby. It is thus necessary to avoid upsetting the baby and/or to be able to soothe them so that the massage is a pleasant experience.

The aim of the invention is particularly that of meeting this need of the prior art.

More specifically, the aim of the invention is that of providing a device for dispensing doses of a cosmetic product enabling favorable conditions for the application of the cosmetic product.

A further aim of the invention is that of providing such a device that is simple to use.

Moreover, an aim of the invention is that of providing such a device that has a simple design.

These aims, as well as others which will emerge hereinafter, are achieved thanks to the invention which relates to a device for dispensing cosmetic product comprising:
a mechanism for dispensing a dose of cosmetic product via a dispensing nozzle;
at least one presence sensor, suitable for detecting a hand movement;
an external surface zone intended to form a hand warmer, and means for heating the external surface zone,
   characterized in that the presence sensor is configured to detect at least:
a first type of hand presence, of a duration between a predetermined first duration t₁ and second duration t₂, and
a second type of hand presence, of a third duration t3 less than the predetermined first duration t₁,
   and in that it further includes electronic management means connected to the presence sensor, to the dispensing mechanism and to the heating means, configured to send:
a dispensing instruction to the dispensing mechanism, when the first presence type is detected, or
a preheating instruction to the heating means, when the second presence type is detected.

Such a device offers the baby particular safety as the masseur or masseuse only uses a single apparatus and can keep one hand in contact with the baby to hold them and prevent them from falling from the massage table.

Indeed, two different presence types can be detected by a single sensor which makes it possible to accustom the user to a single detection location. The user will then merely need to memorize different movements so that the sensor detects the movement and the dispenser acts as desired, either to:
activate dispensing,
start preheating, the user having one hand free during the preheating time.

According to a preferred embodiment:
the first duration is between 2 and 3 seconds, and the second duration is between 5 and 6 seconds, and
the second presence type is between 0.1 s and 1.5 seconds.

Advantageously, the second presence type detected is a succession of detections included within a time interval between 1 and 2 seconds.

Thus, it is possible to clearly differentiate the heating request from the cosmetic product dispensing request. The dispensing of the cosmetic product may then be carried out via a prolonged presence of the user's hand in front of the sensor whereas the preheating may be carried out via the detection of successive movements in front of the sensor.

According to an advantageous embodiment, the second presence type is detected when the presence sensor detects at least two successive presences.

Thus, activating the hand warmer when the sensor detects an accidental presence of a hand is avoided. The two successive passages thus fall within the remit of a voluntary action to activate the hand warmer.

According to a particular embodiment, the device further includes means for the airborne diffusion of an aromatherapy composition, and comprises means for receiving disposable capsules connected to the airborne diffusion means and to the dispensing mechanism, the receiving means receiving disposable capsules of cosmetic preparation ready to be dispensed and/or disposable capsules of aromatherapy composition for airborne diffusion.

According to an advantageous aspect, the device comprises common electronic means for activating the dispensing mechanism and/or the diffusion means.

Advantageously, the device comprises:
means for reading identification codes suitable for being borne by capsules inserted into the receiving means;
electronic means for managing the dispensing of cosmetic product and/or the airborne diffusion of an aromatherapy composition, the electronic management means being coupled with the means for reading identification codes.

Such a dispensing device according to the invention makes it possible to optimize the dispensing conditions of a dose of cosmetic product, as well as the application conditions of the dose of cosmetic product.

Indeed, thanks to the system of disposable capsules, it is easy to prepare the device so that it is ready to dispense a dose.

In addition, thanks for the common electronic means for activating the dispensing mechanism and the diffusion means, the user can receive a dose of cosmetic product and execute a treatment (of the face or body) by applying the dose of cosmetic product dispensed, concomitantly with the airborne diffusion of an aromatherapy preparation.

These electronic means can particularly synchronize the airborne diffusion of the aromatherapy composition with the dispensing of the dose of cosmetic preparation ready to be dispensed, for example by following a predefined or specific program to said cosmetic preparation selected by the user.

For example, the cosmetic product can correspond to the following treatments:
baby changing treatment;
moisturizing treatment;
relaxation treatment, etc.

The aromatherapy can for its part correspond to:
bronchiolitis treatment;
relaxation treatment, etc.

Furthermore, the hygiene of the device is assured through the use of the capsules since the cosmetic product is encapsulated in small quantities which makes it possible to guarantee the efficacy thereof and retain the benefits that it can offer.

According to a preferential embodiment, the device has a spout for dispensing the cosmetic product taking the shape of a goose neck having an end extending downward, the dispensing nozzle being located at the end of the goose neck, and in that the presence sensor is of the contactless type and forms a detection space located in a hollow formed by the goose neck, the hollow being located above a horizontal plane wherein the dispensing nozzle is set.

Thanks to this goose neck shape of the device, the dispensing of a dose of cosmetic product is enhanced.

Indeed, this design forces the user to position their hand below the end of the goose neck while hollowing the hand so as to present the palm ready to receive the product, with the fingers inserted into the hollow of the goose neck, in the presence sensor detection zone. In this way, the hand adopts a particularly suitable shape for collecting the dose of cosmetic product.

In the case where the cosmetic product ready to be dispensed is particularly fluid (oil, milk, etc.), this embodiment of the dispenser (dispensing spout in the shape of a goose neck, detection zone located in the hollow formed by the goose neck, etc.) makes it possible to ensure that the dose will be dispensed correctly and collected in the hollow of the user's hand.

Moreover, such a design makes it possible to hide the sensor to enhance the esthetics of the device and delimit, while keeping same invisible for users, the detection zone which is readily attainable:

### BRIEF DESCRIPTION OF THE DRAWINGS

Further features and advantages of the invention will emerge more clearly on reading the following description of a preferential embodiment of the invention, given by way of illustrative and non-limiting example, and of the appended figures wherein:

FIG. 1 is a schematic representation of the dispensing device according to the invention, according to a perspective view and with the cover thereof open;

FIG. 2 is a schematic representation of the device according to the invention with the cover thereof closed;

FIGS. 3 and 4 are side views of the dispensing device;

FIG. 5 is a schematic representation of the dispensing device according to the invention, according to a front view.

### DETAILED DESCRIPTION OF ILLUSTRATIVE EMBODIMENTS

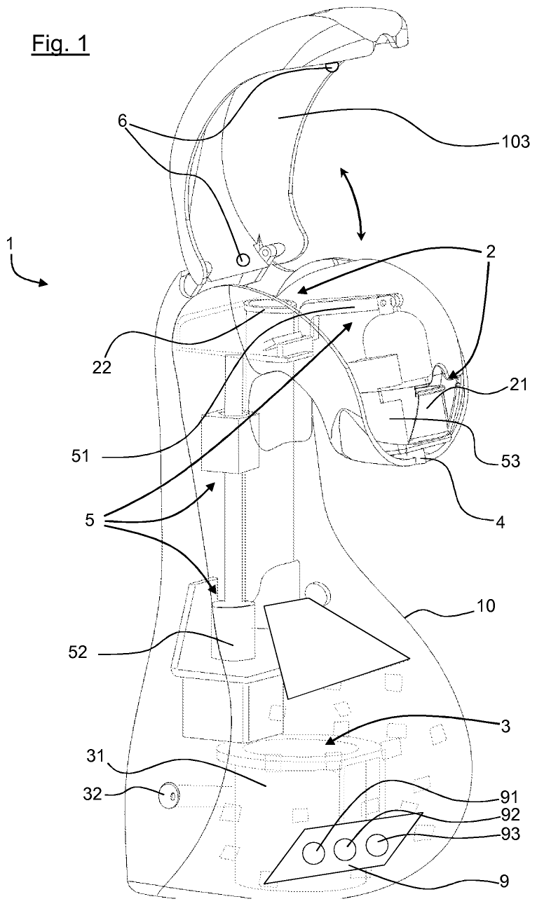

With reference to FIGS. 1 to 5, the dispensing device 1 according to the invention includes a shell 10 having a base in the bottom part and taking, in the top part, the shape of a goose neck.

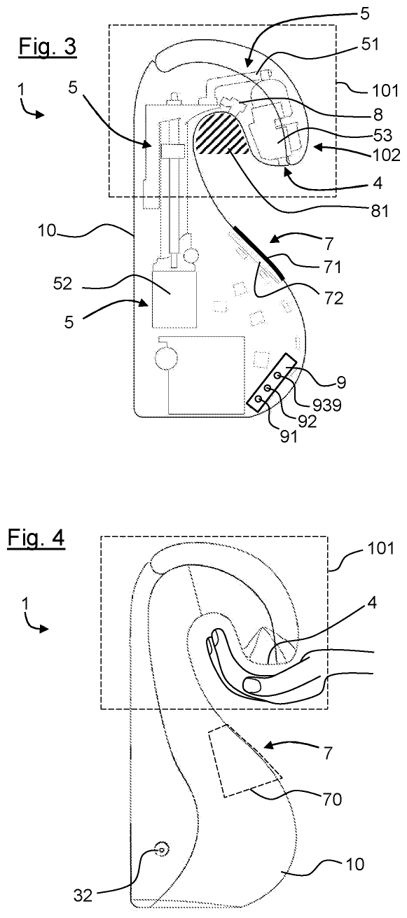

More specifically and as seen in FIGS. 3 and 4, the device according to the invention has in the top part thereof a cosmetic product dispensing spout 101 which takes the shape of a goose neck which has an end 102 extending downwards.

With reference to FIG. 1, the dispensing device according to the invention comprises:
means 2 for receiving disposable capsules of cosmetic preparation ready to be dispensed and/or of aromatherapy composition for airborne diffusion;
means 3 for airborne diffusion of an aromatherapy composition located in one of the capsules;
a dispensing nozzle 4;
a mechanism 5 for dispensing a dose of cosmetic product via the dispensing nozzle 4, from one of the capsules.

The shell 10 of the dispensing device includes a hinged cover 103. This cover 103 is represented in an open position in FIG. 1. In the open position of the cover, the means 2 for receiving disposable capsules are accessible.

With reference to FIG. 1, the receiving means 2 include a first housing 21 (located near the end of the goose neck) for capsules of cosmetic product, and a second housing 22 for capsules of aromatherapy composition.

According to the present embodiment, the capsules intended to be inserted into the machine bear identification codes. As illustrated by FIG. 1, the device 1 comprises means 6 for reading these identification codes when the capsules are inserted into the receiving means. These reading means 6, particularly comprising sensors, are located on the cover 103 and face the capsules inserted into the first housing 21 and into the second housing 22 once the cover has been closed.

According to a further embodiment, the reading means may comprise a single common sensor for reading the codes of both capsules and located outside the device, for example in the vicinity of the dispensing nozzle.

The identification codes of the capsules may for example by barcodes, single-use flash codes, or indeed NFC codes.

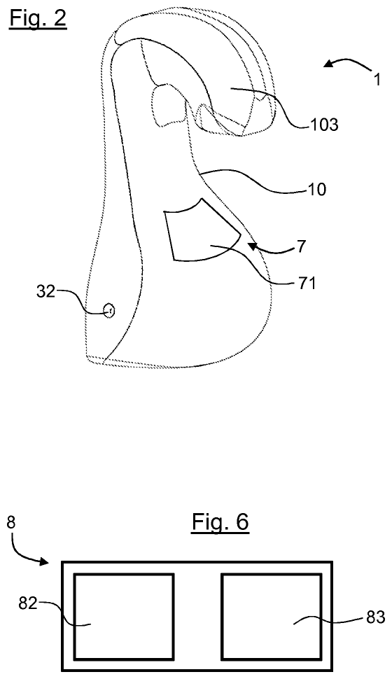

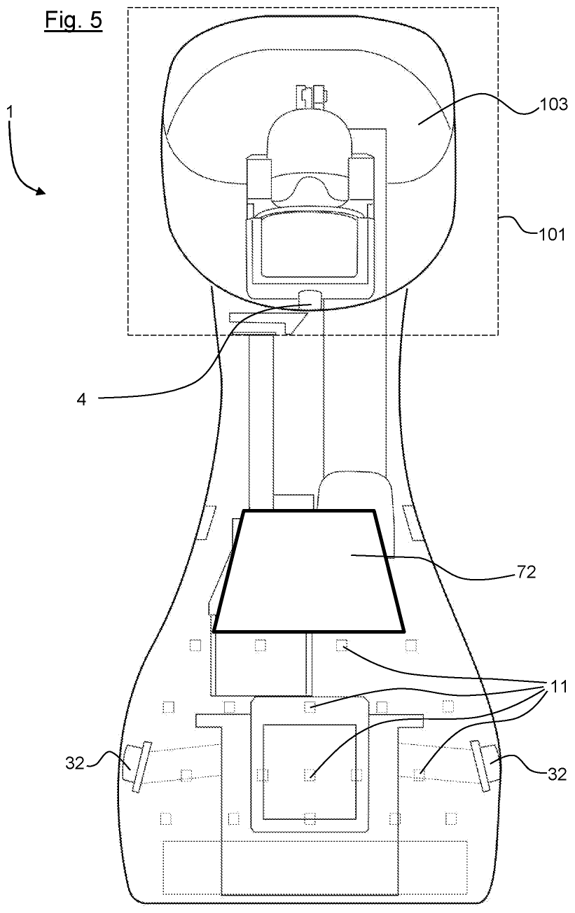

As illustrated by FIGS. 2 to 5, the device 1 comprises:
a hand warmer 7 formed by an external surface zone 71 of the shell 10;
means 72 for heating the external surface zone (represented in FIGS. 3 and 5).

This external surface zone 71 is located on the base of the device and more specifically on the front face of this base.

As illustrated by FIGS. 3 and 4, the device also comprises a “contactless” type presence sensor 8, positioned in the vicinity of the dispensing nozzle 4.

This presence sensor 8 makes it possible to activate the dispensing mechanism 5 upon the presentation of a hand, or of a part of a hand in front of the presence sensor 8.

Once again according to FIGS. 3 and 4, the dispensing nozzle 4 is located at the end 102 of the goose neck, and the “contactless” presence sensor 8 is designed and configured to form a detection space 81 which is located in a hollow formed by the shape of the goose neck, this hollow being located above a horizontal plane wherein the dispensing nozzle 4 is set.

As illustrated in FIG. 4, this particular shape of the dispensing spout 101 and the relative locations of the presence sensor 8 and of the dispensing nozzle 4 force a user seeking to obtain a dose of cosmetic product to curve their hand so that the fingers fit into the detection space 81.

Thanks to this configuration, the user tends to present their hand in an optimized manner to correctly receive the dose of cosmetic product and thus prevent the cosmetic product from flowing next to their hand.

In other words, this contactless presence sensor 8 is positioned set back from and above the dispensing nozzle 4 relative to a direction for inserting a hand to collect a dose of cosmetic product, while being oriented so as to detect the presence of a hand under the dispensing nozzle 4.

This presence sensor 8 does not activate dispensing if a person positions merely a part of their hand just below the dispensing nozzle 4.

According to the principle of the invention, the presence sensor 8 is configured to detect at least:
a first type of hand presence, of a duration between a predetermined first duration t₁ and second duration t₂, and
a second type of hand presence, of a third duration t₃ less than the predetermined first duration t₁

Preferably:
the first duration t₁ is between 2 and 3 seconds, and the second duration t₂ is between 5 and 6 seconds, and
the second presence type is between 0.1 and 1.5 seconds.

More particularly, the second presence type detected is a succession of detections included within a time interval Iₜ between 1 and 2 seconds.

detect this second presence type, the sensor 8 is configured to detect at least two successive presences wherein the total duration, i.e. the duration of a first presence and of a second presence added together, is less than the first duration t₁, but at least between EE and FF seconds. The detector is particularly configured so that these successive detections are performed within the time interval Iₜ mentioned above.

However, although two successive presences are mentioned above, it is also possible to configure the sensor to detect more than two successive presences.

The succession of movement can be carried out by means of a to-and-fro type sweep, i.e. in a first direction then in a second direction opposite the first, or by means of two movements in the same direction.

According to a particular embodiment represented in FIG. 6, the presence sensor 8 comprises a first detection cell 82 and a second detection cell 83 that are contiguous.

By a sensor 8 having two cells 82, 83, the second presence type is detected when the user makes a hand movement corresponding to a sweep, in front of the first cell and the second cell which each individually detect a presence.

The sweep is particular detected when the sensor detects a presence according to a sequence of detection of the first cell, followed by detection of the second cell, and conversely.

According to the present embodiment illustrated by FIGS. 1 and 3, the mechanism 5 for dispensing a dose of cosmetic product comprises:
a piston mechanism 51 actuated by a motor 52;
a circuit 53 for heating the dose to be dispensed.

The heating circuit 53 makes it possible to convey a dose of cosmetic product to be dispensed at body temperature.

The means 3 for airborne diffusion of an aromatherapy composition are of the ultrasound type.

With reference to FIG. 1, on inserting a capsule of aromatherapy composition into the second housing 22 of the receiving means, a needle pierces the capsule to prime the diffusion means 3. On activating the diffusion means 3, droplets of the aromatherapy composition flow from the aromatherapy composition capsule up to a block 31 where they are suspended in the air, in the form of a vapor, using an ultrasound membrane. Fans 32 make it possible to create an air flow to increase the diffusion of the aromatherapy composition suspended in the air.

With reference to FIGS. 1 and 3, the device also comprises an electronic board 9. This electronic board contains:
common electronic means 91 for activating the dispensing mechanism 5 and/or the diffusion means 3;
electronic means 92 for managing the dispensing of cosmetic product and/or the airborne diffusion of an aromatherapy composition;
wireless electronic communication means 93.

Thanks to the common electronic activation means 91, the dispensing of a dose of cosmetic product and the airborne diffusion of an aromatherapy composition can be synchronized, or indeed carried out in a complementary manner to obtain a satisfactory sensory experience particularly easily.

The electronic management means 92 are coupled with the means 6 for reading identification codes. These electronic management means 92 make it possible to adapt the dispensing of cosmetic product and the airborne diffusion of an aromatherapy composition to the products and compositions of the capsules inserted into the device 1.

Indeed, the identification codes enable the configuration means to incorporate, for example, the capacity of the capsules (6 mL, 12 mL, etc.), and the number of doses available in the capsules. According to the identification codes, further parameters of the device according to the invention may be modified such as the heating temperature of the dose of cosmetic product to be dispensed.

A safety device, suitable for locking the device 1, may be fitted to prevent the use of non-conforming cosmetic products and aromatherapy compositions. The identification codes then serve to unlock the device 1.

The wireless electronic communication means 93 enable the dispensing device 1 according to the invention to interact with other electronic devices.

For example, the dispensing device 1 may be paired with a smartphone or an electronic tablet to control the dispensing device 1 remotely, or receive information from the device 1.

The smartphone or the electronic tablet may be provided with a dedicated application for the dispensing device.

According to a practical example, the application may display combinations of aromatherapy compositions and cosmetic products to be used synergistically.

On inserting the relative capsules into the dispensing device 1, the identification codes make it possible to ensure that the correct products have been inserted into the receiving means of the device, and the wireless electronic communication means can then send information relating to these products. The application can then automatically display a demonstration video explaining the method to follow to apply the treatments correctly.

The application can also manage capsule stocks by listing the capsules used, then make it possible to place a new order of capsules accounting for those used.

With reference to FIGS. 2, 4 and 5, the device 1 further includes speakers 10 and light therapy LEDs 11 (illustrated in FIG. 5).

The light therapy LEDs 11 and the speakers 10 can for example be activated during the execution of a particular program (a massage for example). The speakers 10 can then automatically play sounds or relaxing music suitable for the type of massage carried out. Similarly, following the massage, the light therapy LEDs 11 can then follow a precise sequence to optimize the relaxing effect of the massage.

The user can use an electronic tablet on which they have previously installed the information application dedicated to the device 1, and which they have paired with the device 1.

In the application, the user can for example select a massage program that they would like to carry out.

Within the scope of the application of a cosmetic product (a treatment) to a baby by their mother, the application can show the mother a suitable combination including a capsule of relaxing aromatherapy composition and a capsule of ready-to-use cosmetic product.

Following the insertion of the capsules into the device 1, the latter is configured automatically, and a demonstration video can be displayed on the electronic tablet to explain the optimal way to carry out the treatment.

The mother can then begin the treatment by warming her hands using the hand warmer, collecting the dose of cosmetic product heated to body temperature, then starting to carry out the application, for example a massage.

When this treatment is being carried out, the device according to the invention automatically activates the airborne diffusion of the aromatherapy composition, creates atmospheric lighting synchronized with the massage by means of the light therapy LEDs 11 and also diffuses noises or relaxing music also synchronized with the treatment.

In operation, the user can thus choose:
to control the preheating of the heating means 72 of the hand warmer 7 by making a sweeping movement in front of the presence sensor 8 with one hand, or
to control the dispensing mechanism 5 to obtain the dispensing of cosmetic product by the dispensing nozzle 4.

The device 1 according to the invention thus makes it possible to obtain in a simple manner experiential dispensing and application of a cosmetic product while allowing use with one hand to guarantee the baby's safety on the massage table by keeping continuous contact therewith.

Although the present disclosure has been described with reference to one or more examples, workers skilled in the art will recognize that changes may be made in form and detail without departing from the scope of the disclosure and/or the appended claims.

1. A cosmetic product dispensing device comprising:
a mechanism for dispensing a dose of cosmetic product via a dispensing nozzle;
at least one presence sensor, suitable for detecting a hand movement;
an external surface zone forming a hand warmer, and a heater for heating the external surface zone,
wherein the presence sensor is configured to detect at least:
a first type of hand presence, of a duration between a predetermined first duration t₁ and second duration t₂, and
a second type of hand presence, of a third duration t₃ less than the predetermined first duration t₁,
and the presence sensor further includes electronic management circuit connected to the presence sensor, to the dispensing mechanism and to the heater, configured to send:
a dispensing instruction to the dispensing mechanism, in response to the first presence type being detected, and
a preheating instruction to the heater, in response to the second presence type being detected.

2. The cosmetic product dispensing device according to claim 1, wherein the first duration t₁ is between 2 and 3 seconds, and the second duration t₂ is between 5 and 6 seconds.

3. The cosmetic product dispensing device according to claim 1, wherein the second presence type is between 0.1 and 1.5 seconds.

4. The cosmetic product dispensing device according to claim 1, wherein the second presence type detected is a succession of detections included within a time interval Iₜ between 1 and 2 seconds.

5. The cosmetic product dispensing device according to claim 1, wherein the second presence type is detected when the presence sensor detects at least two successive presences.

6. The cosmetic product dispensing device according to claim 1, wherein the presence sensor comprises a first detection cell and a second detection cell and the second presence type is detected when the presence sensor detects a presence according to a sequence of detection of the first cell, followed by detection of the second cell, and conversely.

7. The cosmetic product dispensing device according to claim 1, further comprising a diffuser for airborne diffusion of an aromatherapy composition, and comprises a capsule receiver configured to receive disposable capsules connected to the airborne diffuser and to the dispensing mechanism, the capsule receiver receiving disposable capsules of cosmetic preparation ready to be dispensed and/or disposable capsules of aromatherapy composition for airborne diffusion.

8. The cosmetic product dispensing device according to claim 7, comprising common electronic elements for activating the dispensing mechanism and/or the diffuser.

9. The cosmetic product dispensing device according to claim 8, comprising:
means for reading identification codes suitable for being borne by capsules inserted into the capsule receiver;
wherein the electronic management circuit is further configured to manage dispensing of cosmetic product and/or airborne diffusion of an aromatherapy composition, the electronic management circuit being coupled with the means for reading identification codes.

10. The cosmetic product dispensing device according to claim 1, further comprising a spout for dispensing the cosmetic product and having a shape of a goose neck having an end extending downward and a dispensing nozzle located at an end of the goose neck, and wherein the presence sensor is of a contactless type and forms a detection space located in a hollow formed by the goose neck, the hollow being located above a horizontal plane wherein the dispensing nozzle is set.
